(19) United States
(12) Patent Application Publication
(10) Pub. No.: US 2003/0161938 A1
(43) Pub. Date: Aug. 28, 2003

(54) Composition and method for coating medical devices

(76) Inventor: Bo Johnson, Meersburg (DE)

Correspondence Address: Mitchell P. Brook, Suite 200, 11988 El Camino Real, San Diego, CA, 92130, US

(21) Appl. No.: 10/080,749

(22) Filed: Feb. 22, 2002

(51) Int. Cl. A61L 2/00; A61K 38/06; A61K 31/728; A61K 31/727; A61M 31/00

(52) U.S. Cl. 427/2.28; 427/2.1; 514/54; 514/56; 514/18; 604/500

(57) ABSTRACT

A composition and method for coating medical devices is provided. One embodiment of the present invention employs a coating composition comprising hyaluronic acid and heparin. Another embodiment of the present invention employs a coating composition comprising poly-lysine and heparin. Yet another embodiment of the present invention employs a coating composition comprising hirudin, a peptide and heparin.

### FIELD OF THE INVENTION

The present invention generally relates to surface coatings. More particularly, the invention concerns a composition and method for coating medical devices.

### BACKGROUND OF THE INVENTION

Modern medical procedures routinely involve the insertion of foreign objects into a patient. For example, a variety of intravascular stents and prostheses have been developed for insertion into diseased arteries, thereby inhibiting arterial closure. In addition, many types of medical devices function as substitute blood vessels during open-heart surgery or dialysis.

However, the use of these devices can stimulate adverse body responses, including rapid thrombogenic action and systemic inflammatory reaction. This inflammatory reaction is associated with a variety of post-operative clinical complications, such as increased pulmonary capillary reactions, associated coagulopathies, anaphylactic reactions, and various degrees of organ failure. These complications contribute to the mortality of routine operations, especially in cardiac surgery.

A number of coatings have been developed for medical devices that are intended to promote compatibility between a particular medical device and the environment in which the medical device resides. These biocompatible coatings are generally comprised of several distinct layers that are applied in succession to the device. The coating process may include the use of toxic, or otherwise expensive materials that require special storage and handling procedures. The cost and complexity of the coating process adds to the final production cost of the medical device, increasing health care costs.

Therefore, there exists a need for an inexpensive biocompatible coating that can be applied to medical devices in a simple and safe manner.

### SUMMARY OF THE INVENTION

In order to overcome the deficiencies with known, conventional biocompatible coatings, the present invention is provided. The present invention combines heparin with other active biological substances and thereby enhances blood compatibility as compared to other coatings. This multi-bioactive coating includes antithrombogenic and platelet aggregation inhibition activities along with other activities associated with heparin. The present invention permits alteration of the coating composition to customize the performance of the surface coating for specific needs.

The present invention comprises a base layer that attaches to a medical device surface, or substrate. The base layer may include hyaluronic acid, poly-lysine and a peptide, or a combination of these compounds. A biocompatible compound is then attached to the base layer. The biocompatible compound may include polysaccharides, lipids, proteins, heparin, heparan sulfate, hirudin, aprotinin or a combination of these compounds. The base layer may be applied to the substrate first, or the base layer compound and the biocompatible compound may be mixed together and then applied as a single coating to the substrate.

One embodiment of the present invention employs a coating composition comprising hyaluronic acid and heparin. Another embodiment of the present invention employs a coating composition comprising poly-lysine and heparin. Yet another embodiment of the present invention employs a coating composition comprising hirudin, a peptide and heparin.

The present invention also includes several methods for creating and applying the coating compositions to medical devices.

The present invention provides a coating, and coating method that does not use toxic chemicals or solvents. In one embodiment, the coating can be applied to a medical device in one coating step at room temperature. These and other features and advantages of the present invention will be appreciated from review of the following detailed description of the invention.

### DETAILED DESCRIPTION OF THE INVENTION

In the following paragraphs, the present invention will be described in detail by way of example. Throughout this description, the preferred embodiment and examples shown should be considered as exemplars, rather than as limitations on the present invention. As used herein, “the present invention” refers to any one of the embodiments or equivalents thereof of the invention described herein.

Surgical or other clinical procedures such as dialysis involve extracorporeal blood circulation, where blood is circulated outside the body. The blood contacts the foreign surfaces found on the medical devices that are used during the clinical procedure. For example, some of the literally thousands of medical devices include stents, tubing sets, cardioplegia devices, oxygenators, arterial filters, and blood reservoirs, to name but a few. However, when blood is exposed to non-physiological tissue a systemic “inflammatory reaction” may occur. The inflammatory reaction is associated with a variety of postoperative clinical complications, such as increased pulmonary capillary reactions, associated coagulopathies, anaphylactic reactions, and various degrees of organ failure and may contribute to the mortality in routine operations, especially in cardiac surgery.

The application of biocompatible materials to the foreign surfaces in an extracorporeal circuit or implantable device modifies the normal pattern of blood activation that leads to an inflammatory reaction, and therefore reduces clinical complications. Two known biocompatible compounds are heparin and hirudin. Today there are several heparin coating methods that utilize different techniques in order to bind the heparin to the foreign surface, or substrate.

Present heparin coating methods require the use of toxic chemicals, elevated temperatures, multiple-step procedures or cross-linking compounds. In contrast, the present invention provides a coating method that: does not use toxic chemicals or solvents; can be performed in one coating step; is performed at room temperature; and does not use cross-linking compounds. More specifically, water soluble substances are used in the present invention, and any type of sterilization methods may be used on a medical device coated with the present invention. The coatings of the present invention also are suitable for long term use, as the bonding of the hirudin and the heparin to the medical device surface is essentially irreversible.

One embodiment of the present invention uses heparin in combination with hirudin to provide an ideal biocompatible coating for any type of medical device. As used herein, “medical device” includes any type of device that contacts physiological fluids or tissue.

Another embodiment of the present invention comprises a base layer that attaches to a medical device surface, or substrate. The base layer may include hyaluronic acid, poly-lysine and a peptide, or a combination of these compounds. A biocompatible compound is then attached to the base layer. The biocompatible compound may include polysaccharides, lipids, proteins, heparin, heparan sulfate, hirudin, aprotinin or a combination of these compounds. The base layer may be applied to the substrate first, or the base layer compound and the biocompatible compound may be mixed together and then applied as a single coating to the substrate.

Heparin is a naturally occurring, heavily sulfated polysaccharide widely known for its potent anticoagulant activity. The biological effect of heparin is primarly through interaction with antithrombin III. The heparin molecule contains heavily sulfated residues which allow the polysaccharide to bind to antithrombin with high affinity and thereby accelerates the inactivation of coagulation factors. Preferably, two types of heparin are employed by the present invention: low molecular weight heparin and unfractionated heparin. Other types of heparin may also be used to practice the present invention, such as heparan sulfate.

Hirudin is a substance that is secreted by leeches that prevents blood from clotting. For at least one hundred years, the leech (Hirudo medicinalis) has been effectively used in medical practice where a local anticoagulant effect was needed. Physicians continue to use leech, for example, to overcome isolated microvascular thrombotic problems in reconstructive plastic surgery. Hirudin can now be produced through biotechnology.

Hirudin, which is a pure and homogenous substance, compared to heparin, which is less pure and heterogenous, is the most potent and specific inhibitor of thrombin and has proven to have the strongest anticoagulant and antithrombotic properties known. The mechanism of its inhibitory action is rather simple, involving a direct binding to thrombin without need of any plasma co-factors.

Hirudin and other direct thrombin inhibitors have several advantages over heparin. Hirudin can inhibit thrombin bound to clots or extracellular matrices, which are relatively resistant to heparin. Hirudin does not require antithrombin III as a cofactor, and it is not inhibited by activated platelets, which release platelet factor 4 and other molecules that neutralize heparin. Hirudin can not cause heparin induced thrombocytopenia, which is a decrease in the number of platelets in the blood, resulting in the potential for increased bleeding and decreased clotting ability. REFLUDAN is one type of commercially available recombinant hirudin (REFLUDAN is a registered trademark of Hoechst Marion Roussel Gmbh of Germany).

Conventional bonding techniques for attaching a heparin coating to the surface of a medical device include: ionic bonding, surface grafting, covalent bonding, single point bonding and end point attaching.

There are several drawbacks associated with the above mentioned heparin coating procedures. Mainly, harsh chemicals are employed and toxic synthesis occurs in the chemical/heparin mixture. If the concentration of heparin is low, the blood is exposed to the chemical compound, which is less biocompatible than the surface which it coats. Therefore, the biocompatibility of a device can actually decrease. In addition, ionically bonded heparin will wash out of the device in a very short time.

Furthermore, some medical devices are difficult to coat evenly, and it is difficult to keep the contact times of the different chemicals within the specified limits. Also, the chemicals used with the heparin may have a negative influence on the medical device material, increasing the propensity for cracking of the device.

One feature of the present invention is that heparin is considered or modeled as a polyelectrolyte, which is an ion with multiple charged groups. Therefore, virtually all cationic polypeptides and many anionic polypeptides, especially if they contain the amino acid residues lysine or serine, are capable of binding to heparin.

When treated as a polyelectrolyte, heparin creates polyvalent bindings with ionic interaction due to its polyelectrolytic characteristics and high charge density. At very low pH, heparin creates irreversible conjugates with peptides. Also, at low pH, the solution is bacteriostatic, that is, the low pH solution inhibits the growth or multiplication of bacteria.

One embodiment of the present invention uses natural active surface substances like recombinant polypeptides to bind heparin to the surface of a medical device. These substances can be adsorbed irreversibly to the device surface and form a complex with other polypeptides, and with heparin. The polypeptide adsorption can occur on hydrophilic surfaces as well as on hydrophobic surfaces. The polyelectrolytic characteristic of the polypeptides and the heparin allow a reversible ionic interaction of the substances. In this way, polycovalent bonding can be achieved.

A peptide molecule consists of amino acid residues which have formed a peptide chain whose secondary structure is determined by hydrogen bonds between the peptide units. The conformation of the peptide molecules is determined by bonds between amino acid residues belonging to different parts of the polypeptide chain. These bonds are due to hydrogen, ionic or hydrophobic bonding and to disulphide bridges. Polypeptides cover a large range of molecular weights and have different geometric shapes. Peptide molecules may change the conformation due to physicochemical treatments, as a part of their normal function and due to breaking of the intramolecular bonds.

The most unique feature of a polypeptide is its ability to bind to a wide variety of biological and artificial materials. Most of the associations involve hydrophobic interactions of one type or another. Peptides are reversibly adsorbed in one orientation but may change orientation or conformation to a second irreversible form with the time. It may take time for an adsorbed molecule to develop contact points with the surface, which means that the degree of reversibility or exchangeability of a given molecule decreases with time.

One feature of the present invention is that if a neutral solution of a peptide is titrated with HCl, the titration shows a discontinuity at the iso electric point (IEP), at which point carboxyls abruptly become titrateable. At low pH, an increase in rotational freedom is to be seen. This is due to molecular expansion and increased intramolecular rotation. This facilitates the absorption of the peptide to a surface, for example, the surface of a medical device. Preferably, the peptide includes the amino acid residues of asparagine, glycine and arginine.

At the IEP, the peptide is less soluble and at pH's below the IEP, the peptide forms unsoluble polyelectrolytical complexes with the negatively charged heparin molecule. The lower pH, the more positively charged the peptide molecule becomes. Also, at low pH one has less bacterial growth, which make it easier to work in an aseptic way, and allows for longer use of the peptide solution. In addition, as the contact time between the peptide and the surface to be coated increases, the irreversibility of peptide immobilization also increases. Moreover, there is only a slight difference in adsorption of the peptide between hydrophobic surfaces and hydrophilic surfaces, making calculations of peptide amounts between different types of surfaces easier. Finally, an increase of the peptide concentration results in an increase in adsorbed peptides on the surface.

Preferably, the peptide employed in the present invention is a tetrapeptide with the sequence arginine-glysine-aspargine-serine. Alternatively, a tetrapeptide having the sequence of arginine-glysine-aspargine-lysine may also be employed. In addition, an oligopeptide having a repeating sequence of the above-listed tetrapeptides may also be employed. A number of synthetic and naturally occurring peptides contain these sequences. These peptides inhibit platelet aggregation and may improve the efficacy and potency of thrombolytic therapy. Other suitable peptides may also be used by the present invention. One feature of the above-described peptides is that have the ability to adhere very quickly to a surface. By using these peptides, as a link for hirudin and/or heparin, it is possible to reduce the contact time considerably in order to get an irreversible coating on a medical device, such as a cardioplegia units, oxygenators, or stents.

The above-described peptides also are natural substances and have an affinity to both heparin and hirudin. They also function as a wetting agent by increasing the hydrophilicity of a surface, thereby decreasing the pressure drop in a medical device. They also reduce bacterial adhesion, especially with respect to plastic materials. Finally, these peptides are also relatively cheap compared to other chemicals currently used in medical device coatings.

A unique poly-covalent binding structure makes it possible to coat most materials used in the medical device field. The process uses biological products without the use of harsh chemicals or cross-linkers. The poly-covalent structure involves acylation, alkylation, schiff base formation, thiolation of sulfhydryl residues and sulfonamide bonding.

### COATING SOLUTION EXAMPLE 1

Part I Solution

1) Dissolve 50 milligrams (mg) of hirudin into one liter of sterile water.

2) Adjust the pH to 3.8 by adding HCl to the solution (wait 5 min. and check that the pH is still at 3.8).

3) Dissolve 20 mg of the tetrapeptide in the hirudin solution.

4) Adjust the pH to 3.3 by adding HCl to the solution (wait 5 min. and check that the pH is still 3.3).

Part II Solution:

1) Dissolve 65,000 IU/liter of heparin into a sterile 0.9% NaCl solution (normal saline).

2) Adjust the pH to 2.3 by adding HCl to the solution (wait 5 min. and check that the pH is still 2.3).

Mix coating solution part I and part II together in a closed container. Alternatively, the heparin concentration can be varied in order to govern the hirudin-heparin surface concentration. The above-listed coating solution results in a heparin concentration of 0.25 microgram/cm². Alternative heparin concentrations can range from 0.05 to 0.6 micrograms/cm². The surface concentration of hirudin can range between 0.05 to 0.6 microgram/cm². Both Part I and II solutions can be used for three months without any bacterial growth. Periodically, both solutions should be filtered through a sterile filter and the concentration should be checked. This means that both Part I and II solutions can be reused and therefore, the costs for the coating substances is only what is actually used on the device. This greatly reduces production costs. Another advantage of the above-listed coating solution is that it has an expiration date of at least 2 years.

An alternative embodiment coating may use the tetrapeptide and hirudin as a stand-alone coating.

The above-listed coating solution can be applied to any medical device by performing the following steps: 1) connect clean tubings between the container, roller pump and device; 2) start filling the device by starting the roller pump; 3) check that all air has disappeared in the device; 4) let the coating solution stay in the device for at least 2 hours; 5) empty the solution in the device, preferably by using sterile compressed air; 6) rinse with sterile water, using at least 3 times the liquid volume of the product; 7) check the rinsing solution for residuals of heparin; 8) dry the device, preferably with sterile air.

The above-listed coating solution can be applied to any medical device. This includes, but is not limited to, medical devices constructed from plastics, polymers, polyesters, polyolefins, polycarbonates, polyamides, polyethers, polyethylene, polytetrafluoroethylene, silicone, silicone rubber, rubber, polyurethane, DACRON, TEFLON, polyvinyl chloride, polystyrene, nylon, latex rubber, stainless steel, aluminum alloys, metal alloys, nickel, titanium, ceramics and glass (DACRON and TEFLON are registered trademarks of E.I. du Pont de Nemours and Company of Wilmington, Del.).

### COATING SOLUTION EXAMPLE 2

Instead of mixing Part I and Part II solutions together, the two solutions can be used in consecutive steps. This allows for flexibility in the event you need to lay down a “thicker carpet” of coating on a medical device.

1) Coat with Part I solution for 2 to 16 hours.

2) Drain, rinse with water and blow out excess.

3) Fill with Part II solution and coat for 2 hours.

4) Drain, rinse with water, blow out excess and dry.

### COATING SOLUTION EXAMPLE 3

An alternative embodiment of the present invention uses hyaluronan (hyaluronic acid). Hyaluronic acid is a polysaccharide made up of repeating disaccharide units. Hyaluron is a physiological component that is found in animal connective tissue. Preferably, hyaluronic acid having a molecular weight of about 7 million Dalton is employed, but other molecular weights ranging from 0.5 million Dalton to 30 million Dalton can also be employed.

1) Mix 500 milligrams of hyaluronic acid into one liter of sterile water.

2) Adjust the pH to 2.3 by adding HCl to the solution (wait 5 min. and check that the pH is still at 2.3).

3) Fill the medical device with the hyaluronic acid solution, and let sit for 2 hours.

4) Drain the hyaluronic acid solution from the device.

5) Blow out excess solution with air.

6) Rinse with four times the liquid volume of the device with distilled water.

7) Prepare a Part II heparin solution as described above.

8) Fill the device with the Part II heparin solution, and let sit for 2 hours.

9) Blow out excess solution with air.

10) Rinse with four times the liquid volume of the device with distilled water.

Alternatively, the hyaluronic acid solution and the Part II heparin solution may be mixed together, and applied to a medical device in a single application. Steps 3 and 8 can also be performed at elevated temperatures, such as 40° C. In addition, mixing of the hyaluronic acid solution and the Part II solution may also be performed at elevated temperatures.

### COATING SOLUTION EXAMPLE 4

An alternative embodiment of the present invention uses poly-lysine. Poly-lysine is a non-natural substance that is available in different molecular weights. Preferably, the present invention employs a poly-lysine having a molecular weight of about 300,000 Daltons, but other molecular weights may be used.

1) Mix one gram of poly-lysine into one liter of sterile water.

2) Adjust the pH to 5.5 by adding HCl to the solution (wait 5 min. and check that the pH is still at 5.5).

3) Fill the medical device with the poly-lysine solution, and let sit for 2 hours.

4) Drain the poly-lysine solution from the device.

5) Blow out excess solution with air.

6) Rinse with four times the liquid volume of the device with distilled water.

7) Prepare a Part II heparin solution as described above.

8) Fill the device with the Part II heparin solution, and let sit for 2 hours.

9) Blow out excess solution with air.

10) Rinse with four times the liquid volume of the device with distilled water.

Alternatively, the poly-lysine solution and the Part II heparin solution may be mixed together, and applied to a medical device in a single application. Steps 3 and 8 can also be performed at elevated temperatures, such as 40° C. In addition, mixing of the poly-lysine solution and the Part II solution may also be performed at elevated temperatures.

All of the above-described solutions employ a pH that ranges between 2.0 and 4.0. Other solutions may use a pH that can range between 1 to 6.5. Additionally, the concentrations of hyaluronic acid may vary from about 10 milligrams/liter of water to about 100 grams/liter of water. Similarly, the concentration of poly-lysine may vary from about 10 milligrams/liter of water to about 100 grams/liter of water. Other embodiments of the present invention may employ a pretreatment solution of ammonium peroxydisulfate that would be applied to the surface of the medical device.

Platelet loss has been decreased with the coatings constructed according to the present invention when compared with other coated commercially available products. Beta-thromboglobulin (β-TG) release has also been decreased with the coatings constructed according to the present invention.

Thus, it is seen that a composition and method for coating medical devices is provided. One skilled in the art will appreciate that the present invention can be practiced by other than the preferred embodiments, which are presented in this description for purposes of illustration and not of limitation, and the present invention is limited only by the claims that follow. It is noted that various equivalents for the particular embodiments discussed in this description may practice the invention as well.

What is claimed is:

1. A coating composition comprising:
a base layer selected from a group consisting of hyaluronic acid, poly-lysine and a peptide; and
a biocompatible layer selected from a group consisting of polysaccharides, lipids, proteins, heparin, heparan sulfate, hirudin and aprotinin.

2. The coating composition of claim 1, wherein the hyaluronic acid has a molecular weight that may range between about 50,000 Daltons to about 30 million Daltons.

3. The coating composition of claim 1, wherein the peptide is selected from a group consisting of: tetrapeptides, oligopeptides, peptides having a sequence of arginine-glysine-aspargine-serine, and peptides having a sequence of arginine-glysine-aspargine-lysine.

4. The coating composition of claim 1, wherein the heparin is selected from a group consisting of: low molecular weight heparin, unfractionated heparin and heparin having a molecular weight that may range between 5,000 Daltons and 30,000 Daltons.

5. The coating composition of claim 1, wherein the coating composition is applied to a medical device constructed from at least one material selected from a group consisting of: plastics, polymers, polyesters, polyolefins, polycarbonates, polyamides, polyethers, polyethylene, polytetrafluoroethylene, silicone, silicone rubber, rubber, polyurethane, DACRON, TEFLON, polyvinyl chloride, polystyrene, nylon, latex rubber, stainless steel, aluminum alloys, metal alloys, nickel, titanium, ceramics and glass.

6. A coating composition comprising:
hyaluronic acid; and
heparin.

7. The coating composition of claim 6, wherein the hyaluronic acid has a molecular weight that ranges between about 50,000 Daltons to about 30 million Daltons.

8. The coating composition of claim 6, wherein the hyaluronic acid has a molecular weight of about 7 million Daltons.

9. The coating composition of claim 6, wherein the heparin is selected from a group consisting of: low molecular weight heparin, unfractionated heparin and heparin having a molecular weight that may range between 5,000 Daltons and 30,000 Daltons.

10. The coating composition of claim 6, wherein the coating composition is applied to a medical device constructed from at least one material selected from a group consisting of: plastics, polymers, polyesters, polyolefins, polycarbonates, polyamides, polyethers, polyethylene, polytetrafluoroethylene, silicone, silicone rubber, rubber, polyurethane, DACRON, TEFLON, polyvinyl chloride, polystyrene, nylon, latex rubber, stainless steel, aluminum alloys, metal alloys, nickel, titanium, ceramics and glass.

11. A coating composition comprising:
hyaluronic acid;
heparin; and
hirudin.

12. The coating composition of claim 11, wherein the hyaluronic acid has a molecular weight that ranges between about 50,000 Daltons to about 30 million Daltons.

13. The coating composition of claim 11, wherein the hyaluronic acid has a molecular weight of about 7 million Daltons.

14. The coating composition of claim 11, wherein the heparin is selected from a group consisting of: low molecular weight heparin, unfractionated heparin and heparin having a molecular weight that may range between 5,000 Daltons and 30,000 Daltons.

15. The coating composition of claim 11, wherein the hirudin has a molecular weight of about 6,900 Daltons.

16. The coating composition of claim 11, wherein the coating composition is applied to a medical device constructed from at least one material selected from a group consisting of: plastics, polymers, polyesters, polyolefins, polycarbonates, polyamides, polyethers, polyethylene, polytetrafluoroethylene, silicone, silicone rubber, rubber, polyurethane, DACRON, TEFLON, polyvinyl chloride, polystyrene, nylon, latex rubber, stainless steel, aluminum alloys, metal alloys, nickel, titanium, ceramics and glass.

17. A coating composition comprising:
poly-lysine; and
heparin.

18. The coating composition of claim 17, wherein the poly-lysine has a molecular weight that ranges between about 20,000 Daltons to about 2,000,000 Daltons.

19. The coating composition of claim 17, wherein the heparin is selected from a group consisting of: low molecular weight heparin, unfractionated heparin and heparin having a molecular weight that may range between 5,000 Daltons and 30,000 Daltons.

20. The coating composition of claim 17, wherein the coating composition is applied to a medical device constructed from at least one material selected from a group consisting of: plastics, polymers, polyesters, polyolefins, polycarbonates, polyamides, polyethers, polyethylene, polytetrafluoroethylene, silicone, silicone rubber, rubber, polyurethane, DACRON, TEFLON, polyvinyl chloride, polystyrene, nylon, latex rubber, stainless steel, aluminum alloys, metal alloys, nickel, titanium, ceramics and glass.

21. A coating composition comprising:
hirudin;
a peptide; and
heparin.

22. The coating composition of claim 21, wherein the hirudin has a molecular weight of about 6,900 Daltons.

23. The coating composition of claim 21, wherein the heparin is selected from a group consisting of: low molecular weight heparin, unfractionated heparin and heparin having a molecular weight that may range between 5,000 Daltons and 30,000 Daltons.

24. The coating composition of claim 21, wherein the peptide is a tetrapeptide.

25. The coating composition of claim 21, wherein the peptide is a tetrapeptide having the sequence of: arginine-glysine-aspargine-serine.

26. The coating composition of claim 21, wherein the peptide is a tetrapeptide having the sequence of: arginine-glysine-aspargine-lysine.

27. The coating composition of claim 21, wherein the peptide is an oligopeptide.

28. The coating composition of claim 21, wherein the coating composition is applied to a medical device constructed from at least one material selected from a group consisting of: plastics, polymers, polyesters, polyolefins, polycarbonates, polyamides, polyethers, polyethylene, polytetrafluoroethylene, silicone, silicone rubber, rubber, polyurethane, DACRON, TEFLON, polyvinyl chloride, polystyrene, nylon, latex rubber, stainless steel, aluminum alloys, metal alloys, nickel, titanium, ceramics and glass.

29. A method of creating a coating on an article structured to contact physiological fluids or tissue, the method comprising the steps of:
applying a hyaluronic acid solution to a surface of the article; and
applying a heparin solution to the surface of the article.

30. The method of claim 29, wherein the hyaluronic acid solution has a pH that may range between about pH 1 to about pH 6.5.

31. The method of claim 29, wherein the heparin solution has a pH of about 2.

32. A method of creating a coating on an article structured to contact physiological fluids or tissue, the method comprising the steps of:
applying a solution containing both hyaluronic acid and heparin to a surface of the article.

33. A method of creating a coating on an article structured to contact physiological fluids or tissue, the method comprising the steps of:
applying a poly-lysine solution to a surface of the article; and
applying a heparin solution to the surface of the article.

34. A method of creating a coating on an article structured to contact physiological fluids or tissue, the method comprising the steps of:
applying a coating solution to a surface of the article, the coating solution comprising a mixture of hirudin, a peptide and heparin.

35. The method of claim 34, wherein the peptide is a tetrapeptide.

36. The method of claim 34, wherein the peptide is a tetrapeptide having the sequence of: arginine-glysine-aspargine-serine.

37. The method of claim 34, wherein the peptide is a tetrapeptide having the sequence of: arginine-glysine-aspargine-lysine.
